(19) United States
(12) Patent Application Publication
(10) Pub. No.: US 2026/0041504 A1
(43) Pub. Date: Feb. 12, 2026

(54) END EFFECTORS FOR OPHTHALMIC SURGERY SETUP ROBOT

(71) Applicant: Alcon Inc., Fribourg (CH)

(72) Inventors: Patrick W. TERRY, Goleta, CA (US); Satish YALAMANCHILI, Lake Forest, CA (US); Nanhong LOU, Fort Worth, TX (US)

(21) Appl. No.: 19/295,424

(22) Filed: Aug. 8, 2025

Related U.S. Application Data
(60) Provisional application No. 63/681,603, filed on Aug. 9, 2024.

(51) Int. Cl. A61B 34/30; A61B 50/00; A61B 50/24; A61B 50/30; A61B 90/00; A61F 9/007

(52) U.S. Cl. CPC A61B 34/30; A61B 50/24; A61B 50/30; A61F 9/007; A61B 2050/002; A61B 2090/064; A61B 2090/0805

(57) ABSTRACT

In certain embodiments, a system includes a first robotic arm and a controller. The first robotic arm includes a first end effector and a second end effector. The first end effector is configured to hold and manipulate a surgical package containing an item. The second end effector includes a sensor configured to transmit sensor data. The controller is coupled to the first end effector and the second end effector, and is configured to control the first end effector to present the surgical package to the sensor of the second end effector, command the second end effector to acquire the sensor data, determine, based on the sensor data, a type of item within the surgical package.

### CROSS-REFERENCE TO RELATED APPLICATIONS

This application claims priority to and benefit of U.S. Provisional Patent Application No. 63/681,603, filed on Aug. 9, 2024, the content of which is hereby expressly incorporated by reference in its entirety as if fully set forth below and for all applicable purposes.

### BACKGROUND

The present disclosure relates to ophthalmic surgery.

Common ophthalmic surgical treatments include cataract surgery, glaucoma treatments, retinal membrane peeling, vitrectomy, and retinal reattachment. The structures of the eye are extremely small and delicate. Ophthalmic surgery is therefore extremely sophisticated and becoming an ophthalmic surgeon requires many years of training. The time of an ophthalmic surgeon spent in the operating room is therefore a very valuable resource.

### SUMMARY

In certain embodiments, a system includes a first robotic arm and a controller. The first robotic arm includes a first end effector and a second end effector. The first end effector is configured to hold and manipulate a surgical package containing an item. The second end effector includes a sensor configured to transmit sensor data. The controller is coupled to the first end effector and the second end effector, and is configured to control the first end effector to present the surgical package to the sensor of the second end effector, command the second end effector to acquire the sensor data, determine, based on the sensor data, a type of item within the surgical package.

In certain embodiments, the system further includes a second robotic arm. The second robotic arm includes a third end effector and a fourth end effector. The third end effector is configured to hold and manipulate the surgical package, and the fourth end effector is configured to open the surgical package. The controller is further configured to control the third end effector to present the surgical package to the fourth end effector, control the fourth end effector to open the surgical package based on the type of item, the opening portion of the surgical package, and the location of the item, and control the third end effector to release the item from the surgical package.

### BRIEF DESCRIPTION OF THE DRAWINGS

FIG. 1A is schematic diagram of an operating environment including a setup robot, in accordance with embodiments of the present disclosure.

FIG. 1B illustrates an example setup robot including a robotic arm mounted to a guide rail, in accordance with embodiments of the present disclosure.

FIG. 1C illustrates an example setup robot including two robotic arms mounted to a guide rail, in accordance with embodiments of the present disclosure.

FIG. 1D illustrates another example setup robot including two robotic arms mounted to a guide rail, in accordance with embodiments of the present disclosure.

FIG. 1E is schematic diagram of an operating environment including one or more setup robots, in accordance with embodiments of the present disclosure.

FIG. 1F illustrates an example setup robot including a robotic arm, in accordance with embodiments of the present disclosure.

FIG. 1G illustrates a tray holding supplies for an ophthalmic treatment, in accordance with embodiments of the present disclosure.

FIG. 1H illustrates components for operating a setup robot, in accordance with embodiments of the present disclosure.

FIG. 2A is a diagram illustrating an example end effector for identifying an item in a surgical package, in accordance with embodiments of the present disclosure.

FIG. 2B is a diagram illustrating another example end effector for identifying an item in a surgical package, in accordance with embodiments of the present disclosure.

FIG. 2C is a diagram illustrating another example end effector for identifying an item in a surgical package, in accordance with embodiments of the present disclosure.

FIG. 3A is a diagram illustrating example end effectors for opening a surgical package, in accordance with embodiments of the present disclosure.

FIG. 3B is a diagram illustrating releasing an item from a surgical package, in accordance with embodiments of the present disclosure.

FIG. 4A is a diagram illustrating another example end effector for opening a surgical package, in accordance with embodiments of the present disclosure.

FIG. 4B is a diagram illustrating opening a surgical package using the end effector of FIG. 4A, in accordance with embodiments of the present disclosure.

FIG. 5A is a diagram illustrating another example end effector for opening a surgical package, in accordance with embodiments of the present disclosure.

FIG. 5B is a diagram illustrating opening a surgical package using the end effector of FIG. 5A, in accordance with embodiments of the present disclosure.

FIG. 6 depicts process an example flow diagram presenting functionality associated with example end effectors, in accordance with embodiments of the present disclosure.

### DETAILED DESCRIPTION

In certain embodiments, a system for ophthalmic surgery may include one or more patient stations configured to facilitate the performance of ophthalmic procedures by the surgeon. Each patient station may include a patient support (such as a bed, chair, etc.), a surgical microscope mounted to an adjustable support, a surgical table for supporting a tray with items that will be used during the ophthalmic procedure, and a disposal station for disposing, storing or processing used items from the tray. A supply area (such as a table, bench, cabinet, etc.) stores the tray (or trays) that will be used during the ophthalmic procedure.

The system also includes cameras positioned in various locations of the operating environment to provide views of the patient stations and the supply area, a setup robot, and a controller that is coupled to the cameras and the setup robot. Generally, the setup robot includes a robotic arm with a camera and an end effector. The controller is configured to perform various functions related to the ophthalmic procedure, such as processing image data that are received from the cameras, controlling the setup robot before, during, and after the ophthalmic procedure, etc. For example, the controller may command the setup robot to prepare the patient station for the ophthalmic procedure by transferring one of the trays from the supply area to the surgical table. In other examples, the controller may command the setup robot to pass items to the surgeon from the tray on the surgical table, place items received from the surgeon back on the tray on the surgical table, discard items received from the surgeon into the disposal station, transfer the tray from the surgical table to the supply table after the ophthalmic procedure is completed, etc.

The items needed for the ophthalmic procedure may include instruments, consumable products, structures to be implanted, etc. These items arrive at the surgical facility in sealed packages to protect the sterility of each item prior to the ophthalmic procedure. Unfortunately, surgical staff must manually load each tray by opening each scaled package and placing the item on the tray. The required motions that surgical staff perform when identifying and opening these sealed packages are technically complex, and surgical staff training bottlenecks may prevent the optimum efficiency. Additionally, the inventory of the items placed on the trays is not automatically tracked, and may be dependent upon the diligence of the surgical staff. Because high-volume ophthalmic procedure facilities require a large number of surgical staff, staffing shortages for scrub-techs, nurses, etc., impact the efficiency of these facilities. Staffing shortages invariably lead to the need for surgical staff to work overtime hours, which causes stress and high mental strain.

Certain embodiments of the present disclosure advantageously replace the manual loading process for each tray with a robotic-based process that reduces surgical staffing requirements, does not require training of new surgical staff or re-training of current surgical staff, and can automatically track inventory. In certain embodiments, a system for ophthalmic surgery includes robotic arms with multiple end effectors that interact with surgical packages containing items used during ophthalmic surgery in order to identify the items, open the surgical packages, and release the items from the surgical package (such as onto the tray).

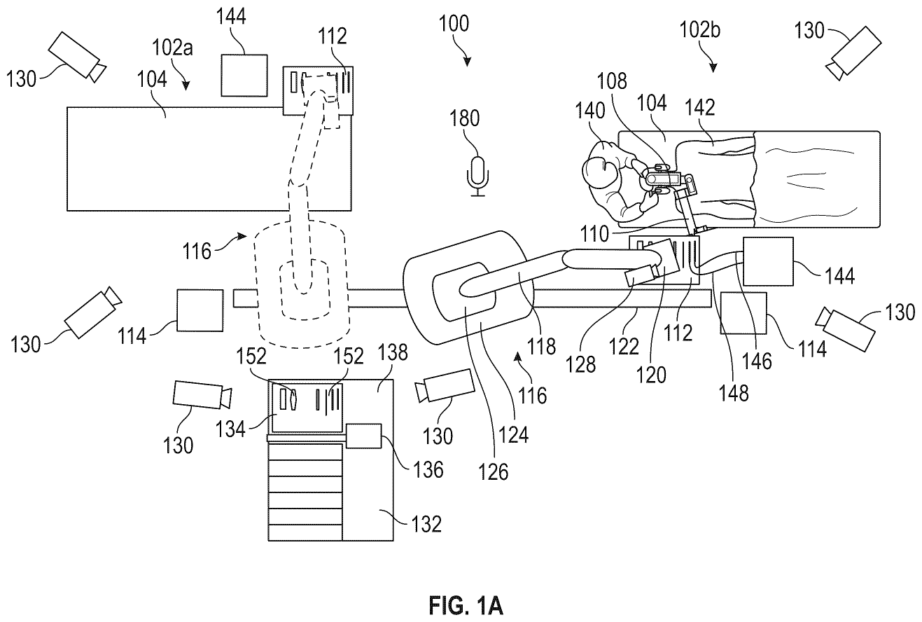

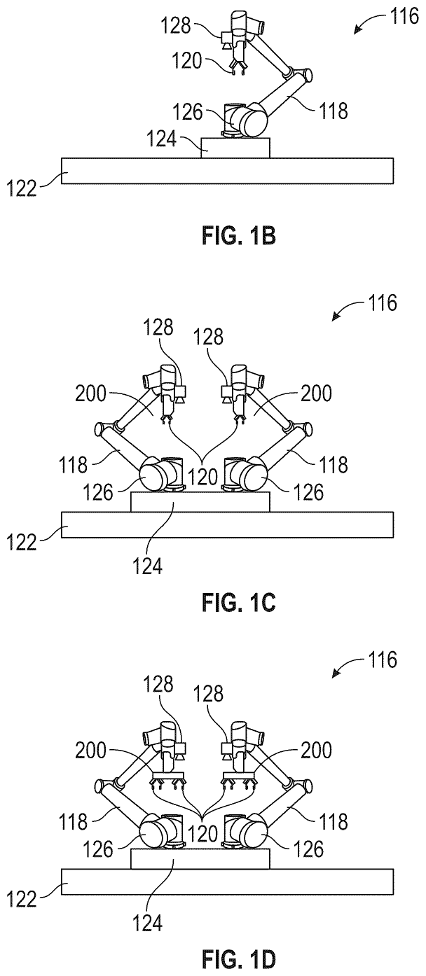

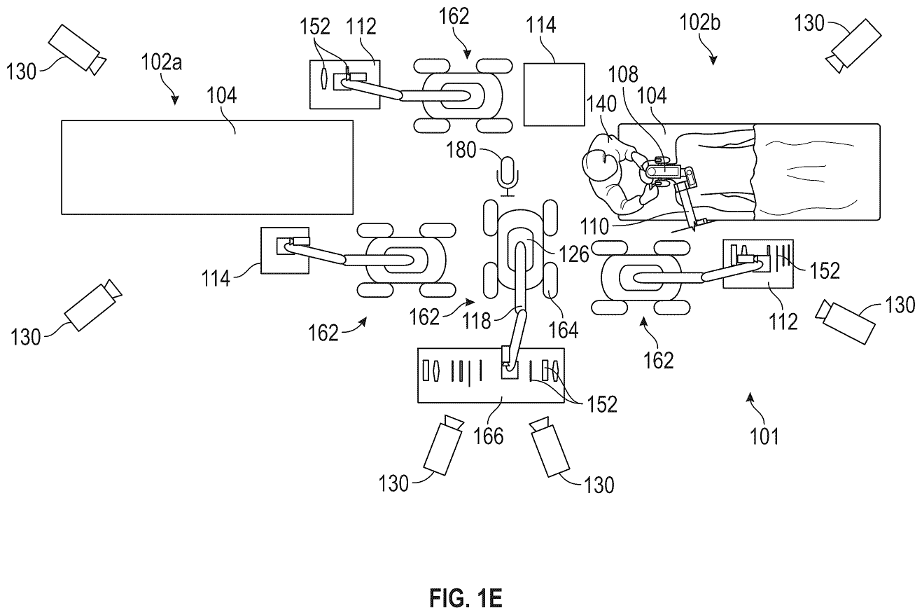

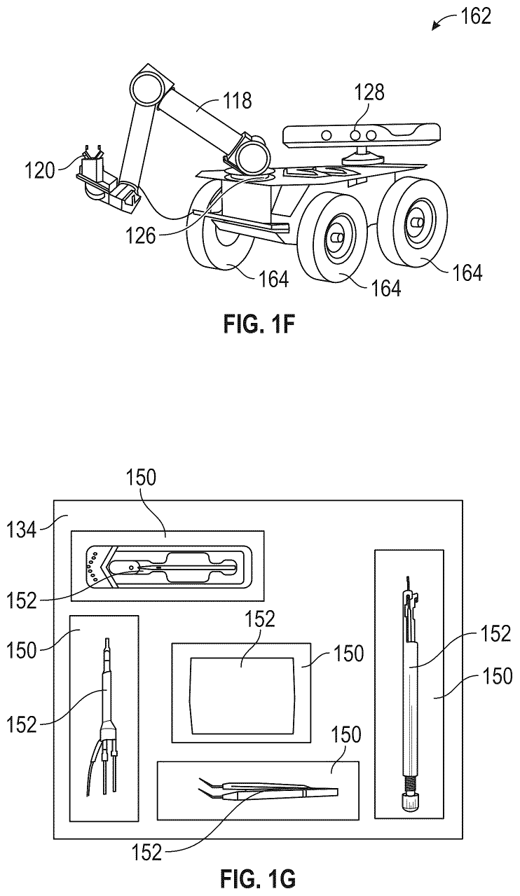

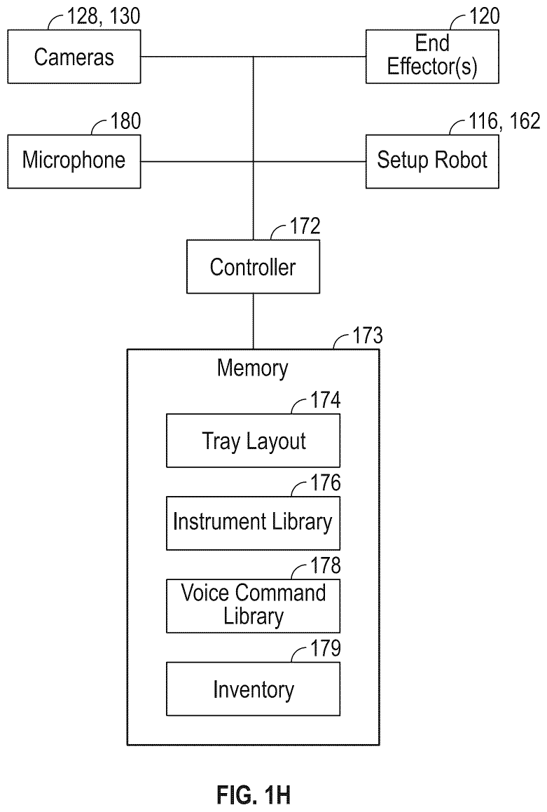

Referring to FIGS. 1A, 1B, IC, ID an operating environment 100 includes two or more stations 102a, 102b, and each station 102a, 102b includes a patient support 104 for supporting a patient 106, such as a bed, chair, or other type of support. Although the system and methods disclosed herein are advantageously used with multiple stations 102a, 102b, a single station 102a may also be used.

Each station 102a, 102b may include other equipment, such as an ophthalmic microscope 108 mounted to an adjustable support 110. In some embodiments, a single ophthalmic microscope 108 may be used, and the adjustable support 110 facilitates movement of the ophthalmic microscope 108 between station 102a and station 102b.

Each station 102a, 102b may include a surgical table 112 and a disposal station 114. The surgical table 112 provides a surface for supporting surgical supplies, such as a tray containing the items needed during the ophthalmic procedure. The disposal station 114 may include a waste bin, an autoclave, a collection bin for items to be sanitized elsewhere, a hazardous material disposal bin, a receptacle for storing or processing used surgical supplies, or a combination of these containers.

One or more setup robots 116 may be positioned in the operating environment 100. In certain embodiments, a single setup robot 116 may be used, and the setup robot 116 may prepare one station 102a (102b) while the other station 102b (102a) is in use. FIG. 1A depicts a setup robot 116 in a first position supporting station 102a (solid line), and in a second position supporting station 102b (dotted line). In other embodiments, a setup robot 116 may be provided for each station 102a, 102b, and FIG. 1A depicts a setup robot 116 supporting station 102a (solid line), and another setup robot 116 supporting station 102b (dotted line).

In the embodiments depicted in FIGS. 1A, 1B, IC, ID the setup robot 116 includes a robotic arm 118. The robotic arm 118 may be a serial robotic arm, and may have 4 to 8 (or more) degrees of freedom. The degrees of freedom may be sufficient to position the end effector 120 of the robotic arm 118 at various three-dimensional positions and orientations within the working envelope of the robotic arm 118. The degrees of freedom may include one (or more) degrees of freedom of the end effector 120.

The end effector 120 may be a gripper, a process tool, a sensor, etc. The end effector 120 may also be a combination of a gripper and a process tool, a gripper and a sensor, a process tool and a sensor, etc. A gripper grabs and manipulates objects. A process tool performs a process on an object, such as fastening, welding, grinding, sanding, cutting, etc. A sensor provides measured data associated with an object or the surrounding environment, such as ultrasonic data, proximity data, force data, torque data, light data, etc.

In certain embodiments, the end effector 120 may be directly attached to the end or wrist of the robotic arm 118 using fasteners, such as screws, bolts, etc. In some embodiments, the end effector 120 may be directly attached to a mechanical coupling (such as a bracket, etc.) using fasteners, and the mechanical coupling may be directly attached to the end of the robotic arm 118 using fasteners. Alternatively, the mechanical coupling may be attached to an adapter plate that is directly attached to the end of the robotic arm 118.

In certain embodiments, the end or wrist of the robotic arm 118 may include a releasable coupling, such as a universal joint, a universal coupling, a quick change coupling, etc., that is configured to receive and secure different types of end effectors 120 to the robotic arm 118. For example, the releasable coupling may cooperate with a robotic arm mount located on the end effector 120 to secure the end effector 120 to the robotic arm 118. Advantageously, the operator (such as the surgeon, the surgeon's assistant, etc.) may select and secure a particular end effector 120 to the robotic arm 118 based on the type of item to be identified in a surgical package (such as end effectors 230, 240, 250 discussed below), or the type of surgical package to be opened (such as end effectors 260, 270, 280, 290 discussed below).

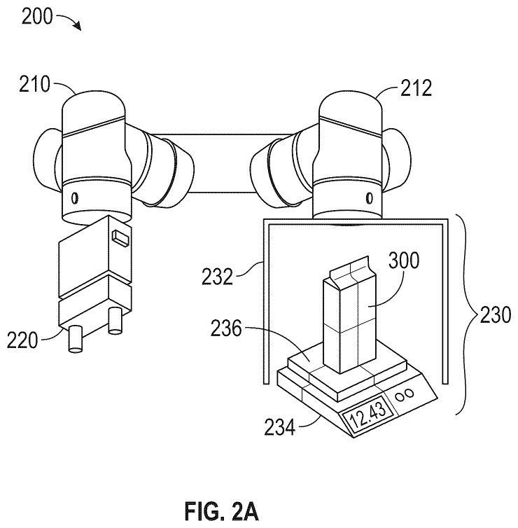

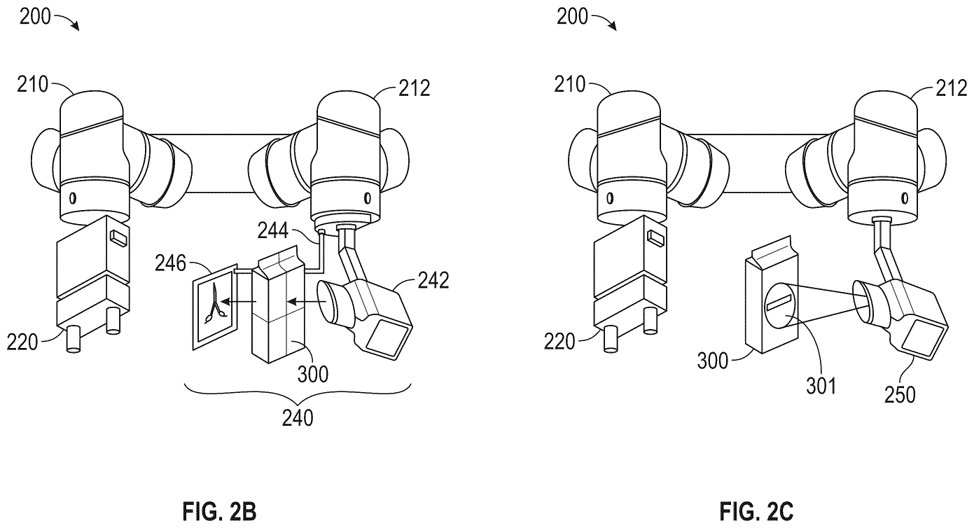

In the embodiment depicted in FIG. 1B, the end effector 120 may be a gripper for holding and releasing objects (such as gripper 220 depicted in FIG. 2A). The gripper may be any structure configured to hold, manipulate, and release objects. The gripper may be configured to hold, manipulate, and release objects that have structures or features specifically designed to cooperate with the gripper. The gripper may be a mechanical gripper (such as a parallel-jaw gripper, three-finger gripper, a needle gripper, etc.), a magnetic or electromagnetic gripper, a pneumatic gripper, a vacuum gripper, an electric gripper, a hydraulic gripper, etc. In certain embodiments, one or more docking structures may be arranged in pre-set locations on the floor of operating environment 100. Each docking station may be configured to receive at least a portion of the end effector 120 to secure the end effector 120 to the docking station,

The setup robot 116 may include a rail actuator 124 that is mounted a rail 122 (such as a linear rail, etc.). The rail actuator 124 may include a motor and a gear, wheel, or other structure that engages the rail 122. The gear is driven by the motor to translate the setup robot 116 to different positions along the rail 122. The rail 122 may be mounted to a floor, ceiling, or wall of the operating environment 100. Multiple rails and corresponding actuators may be used to implement a two-dimensional or three-dimensional translating gantry. In other embodiments, the rail actuator 124 does not include a motor, and the setup robot 116 may be manually translated along the rail 122 and locked in position.

Precise positioning of the end effector 120 may be performed in various ways. In certain embodiments, a kinematic state of the setup robot 116 and a known mapping of objects in the operating environment 100 is used along with obstacle detectors to position the end effector 120. Cameras or other local positioning system (LPS) may not be needed. In certain other embodiments, one (or more) cameras 128 may mounted to the robotic arm 118 on or near the end effector 120. For example, a camera 128 may be mounted within 15 cm of, and rigidly coupled to, the end effector 120. Images from the camera 128 may be processed to determine the location and orientation of the end effector 120, and used as feedback to control the robotic arm 118. In certain other embodiments, several cameras 130 may be distributed around the operating environment 100 in order to view the end effector 120. Visual markers may be attached to the end effector 120 and the robotic arm 118 to facilitate image recognition in the image data from the cameras 130. Image data from the cameras 130 may then be processed to determine the position and orientation of the end effector 120. The position and orientation data are used to control the robotic arm 118 to achieve a desired position and orientation of the end effector 120.

A supply area 132, such as a table, bench, cabinet, etc., may be positioned in the operating environment 100 within the operating envelope of the setup robot 116. The supply area 132 may support one or more trays 134. Each tray 134 may be loaded with items needed during an ophthalmic procedure. In some scenarios, more than one tray 134 may used for a single ophthalmic procedure. The supply area 132 may include a gate 136 that allows trays 134 to drop, slide, or otherwise move into a pickup area 138. For example, trays 134 may be arranged according to a schedule of ophthalmic procedures such that each tray 134 may be retrieved by the setup robot 116 for each ophthalmic procedure in the schedule. In another example, during an ophthalmic procedure on a patient 106 in station 102b, the setup robot 116 may retrieve a tray 134 for the next ophthalmic treatment in station 102a, and place the tray 134 onto the surgical table 112 of station 102a.

In some embodiments, each station 102a, 102b may include a console 144 that provides ports for connecting tubes 146 for conducting vacuum pressure or infusion fluid, connecting electrical lines 148 for supplying power, or other types of ports. The setup robot 116 may prepare each station 102a, 102b for a surgery by connecting each tube 146 and electrical line 148 between the console 144 (or other housing for a port) and an instrument, such as an instrument in the tray 134.

In the embodiment depicted in FIG. 1C, the setup robot 116 includes a first robotic arm 118 and a second robotic arm 118. Each robotic arm 118 may be a serial robotic arm, and may have 4 to 8 (or more) degrees of freedom. Each robotic arm 118 may have an end effector 120. The degrees of freedom of the robotic arms 118 may be sufficient to position each end effector 120 at various three-dimensional positions and orientations within the working envelope of the respective robotic arm 118. The end effector 120 may be a gripper, a tool changer, etc. For example, the gripper may be a mechanical gripper (such as a parallel-jaw gripper, three-finger gripper, a needle gripper, etc.), an electromagnetic gripper, a pneumatic gripper, a hydraulic gripper, etc. The end effector 120 may also be an imaging device that includes a sensor, as described below. In certain embodiments, the end effector 120 for the first robotic arm 118 may be a gripper, and the end effector 120 for the second robotic arm 118 may be an imaging device.

In the embodiment depicted in FIG. 1D, the setup robot 116 includes a first robotic arm 118 and a second robotic arm 118. Each robotic arm 118 may be a serial robotic arm, and may have 4 to 8 (or more) degrees of freedom. Each robotic arm 118 may have a dual end effector arm 200. Each dual end effector arm 200 may include two end effector arms 210, 212 (see FIG. 2A), and each end effector arm 210, 212 may have an end effector 120. Each end effector arm 210, 212 may have 1 to 4 (or more) degrees of freedom. The degrees of freedom of the robotic arms 118 and the end effector arms 210, 212 may be sufficient to position each end effector 120 at various three-dimensional positions and orientations within the working envelope of the respective robotic arm 118. The end effector 120 may be a gripper, a tool changer, etc. For example, the gripper may be a mechanical gripper (such as a parallel-jaw gripper, three-finger gripper, a needle gripper, etc.), an electromagnetic gripper, a pneumatic gripper, a hydraulic gripper, etc.

FIGS. 1E, IF illustrates an alternative operating environment 101. The operating environment 101 may include stations 102a, 102b, patient supports 104, surgical microscope 108, support 110, surgical table 112, one or more disposal stations 114, cameras 128, 130, and one or more setup robots 162. In certain embodiments, the setup robot 162 may include a single robotic arm 118 with an end effector 120 and a camera 128 (as depicted in FIG. 1B). In some embodiments, the setup robot 162 may include two robotic arms 118, and each robotic arm may include an end effector 120 and a camera 128 (as depicted in FIG. 1C). In some embodiments, the setup robot 162 may include two robotic arms 118, each robotic arm 118 may have a dual end effector arm 200 and a camera 128, each dual end effector arm 200 may have two end effector arms 210, 212 (see FIG. 2A), and each end effector arm may have an end effector 120 (as depicted in FIG. 1D).

The base 126 of the robotic arm 118 may be mounted to a body with actuated floor engaging members 164 configured to move the base 126 in one or more dimensions along a floor of the operating environment 101. The floor engaging members 164 may be wheels, treads, articulated legs, or any other approach for inducing translational motion across a flat surface. One or more cameras 128 may be mounted to a fixed or rotating arm for navigation within operating environment 101.

The setup robot 162 (as well as the setup robot 116 as described above) may be used without the benefit of pre-packed trays 134. For example, an end effector 120 and camera 128 of the robotic arm 118 may be used to identify, grasp, and place items 152 that are in a supply area 166 but not necessarily positioned in trays 134. The items 152 may be in bins with other items 152 of the same type. The items 152 may be in dispensers configured to interface with the end effector 120. The items 152 may also be laid out on a flat surface. Where a disposal station 114 is an autoclave or other type of cleaning device, the disposal station 114 may also function as a supply area 132 from which items 152 are retrieved using the setup robot 162 following cleaning and disinfection.

In the embodiment depicted in FIG. 1G, each tray 134 support a number of items 152 in respective recesses 150. Each item 152 may be an instrument, a consumable product, a structure to be implanted, or other item for use during an ophthalmic treatment. In other embodiments, the tray 134 does not include recesses 150, and the items 152 simply rest on the surface of the tray 134.

There are many items 152 that may be used to perform any number of ophthalmic treatments such as phacoemulsification and IOL placement, vitrectomy, glaucoma surgery, retinal attachment, refractive surgery (laser-assisted in situ keratomileusis (LASIK), small incision lenticule extraction (SMILE), implantable contact lens (ICL), etc.), or other ophthalmic treatments. A non-limiting list of example items 152 includes a sideport incision instrument, topical or injected anesthesia and corresponding syringe or other dispenser, cystotome, balanced salt solution (BSS), centurion handpiece, silicone, metal, or polymer irrigation/aspiration (I/A) tip, ophthalmic viscosurgical device, tryphan blue and applicator, forceps, OVD removal tool, fluidics management system (FMS) pack, metal or plastic handpiece, pre-loaded disposable or reusable intraocular lens (IOL) injector, primary incision instrument, irrigation/hydrodissection tool, sutures, drapes, etc.

In certain embodiments, the layout of the tray 134 is known such that the controller for the setup robot 116 does not require visual recognition of the item within each recess 150. Instead, the controller may simply position the end effector 120 (such as a gripper) at a known location of a recess 150 containing an item 152 and lift the item 152 from the tray. In certain embodiments, the items 152 and the recesses 150 may include markings, text, or other computer-readable symbols that may be used by the controller to identify the item 152 located in a particular recess 150.

In the embodiment depicted in FIG. 1H, the setup robot 116, the end effector(s) 120, the cameras 128, 130, and the microphone 180 may be coupled to a controller 172 using wired connections (such as USB, Ethernet, etc.) or wireless connections (such as Bluetooth, WiFi, etc.). The controller 172 may be coupled to a memory 173. The controller 172 may be a general-purpose computer, programmable logic controller (PLC), or other electronic device programmed to perform the functions ascribed herein to the controller 172. Memory 173 may store instructions to be executed by controller 172, data, and other information, such as a tray layout 174, an instrument library 176, a voice command library 178, an inventory 179, etc.

The controller 172 may access a tray layout 174 for each tray 134 to be used for each ophthalmic procedure. The tray layout 174 may include an identifier of each tray 134 enabling the tray 134 to be identified, such as a marking, text, or other symbol affixed to the tray 134. The tray layout 174 may include a specification of the location of recesses 150 and an identifier of the item 152 positioned within each recess 150.

In some embodiments, to further facilitate the identification of items 152, the controller 172 may access the instrument library 176. For each type of item 152, the instrument library 176 may include such information as a marking, text, or other symbol that uniquely identifies the item 152, a position of each item 152 in each tray layout 174, a three-dimensional model of the item 152 enabling the items 152 to be identified in images from the cameras 128, 130, one or more two-dimensional images from different angles, or other data to facilitate machine identification of each type of item 152.

The controller 172 may be configured to evaluate images from cameras 128, 130, determine that the tray 134 in the pickup area 138 is the correct tray for of a scheduled ophthalmic procedure by detecting identification data in the images, cause the setup robot 116 to grab the tray 134 and move the tray 134 to the surgical table 112 of a station 102a, 102b for which the ophthalmic treatment is scheduled. As shown in FIG. 1A, the range of motion of the setup robot 116 along the rail 122 enables the setup robot 116 to place a tray 134 on the surgical tables 112 of both stations 102a, 102b.

The controller 172 may be configured to identify used items 152 returned to the tray 134 in the images from cameras 128, 130, and move the used items 152 to a disposal station 114 to be disposed of or disinfected for subsequent use.

In certain embodiments, the controller 172 may be configured to grasp an item 152 from a tray 134 and pass the item 152 to the surgeon 140 in response to a voice command. For example, the controller 172 may access the voice command library 178, which includes voice commands that specify an action and an identifier of an item 152, such as an action to pass an item 152 from a tray 134 to a surgeon, an action to receive an item 152 from a surgeon and pass the item 152 to the disposal station 114, etc. The microphone 180 may be centrally-located in the operating environment 100 to detect the voice commands of the surgeon 140. In other embodiments, each station 102a, 102b may have a corresponding microphone 180.

FIG. 2A is a diagram illustrating an end effector 230 for identifying an item in a surgical package 300, in accordance with embodiments of the present disclosure.

In certain embodiments, the dual end effector arm 200 includes an end effector arm 210 and an end effector arm 212. The dual end effector arm 200 is coupled to the end of the robotic arm 118 of setup robot 116, such as the first robotic arm 118 (depicted in FIG. 1B, 1D) or the second robotic arm 118 depicted in FIG. 1D.

The gripper 220 is an end effector that is coupled to the end effector arm 210. The gripper 220 is configured to hold, manipulate, and release the surgical package 300, and is coupled to the controller 172. For example, the gripper 220 may be a mechanical gripper (such as a parallel-jaw gripper, three-finger gripper, a needle gripper, etc.), an electromagnetic gripper, a pneumatic gripper, a hydraulic gripper, etc.

The end effector 230 is coupled to the end effector arm 212. Generally, the end effector 230 includes a sensor that is configured to transmit sensor data to the controller 172. More particularly, the end effector 230 includes a scale 234 that includes a number of load cells 236, such as 3 load cells 236, 4 load cells 236, 8 load cells 236, etc. The load cells 236 may be pancake load cells with strain gages, etc. The scale 234 is configured to transmit load cell data to the controller 172. The end effector 230 may include a frame 232 (such as a basket, etc.) to support the surgical package 300 in the center of the scale 234.

In certain embodiments, the scale 234 includes 4 load cells 236 (as depicted in FIG. 2A). Each load cell 236 is located at a corner of the scale 234 and at a distance Xi and Yi from the origin of the scale 234. Each load cell 236 provides a signal that represents a portion of the weight (Wi) of the surgical package 300. The total weight (WT) of the surgical package 300 is simply the sum of the weights (Wi) measured by the load cells 236, as provided by Equation 1:

$${W}_{T} = {W}_{1} + {W}_{2} + {W}_{3} + {W}_{4} \qquad Eq . 1$$

The center-of-gravity (CoG) of the surgical package 300 is represented by an XCoG coordinate and a YCoG coordinate relative to the origin of the scale. The XCoG may be calculated using Equation 2, while the YCoG may be calculated using Equation 3:

$${X}_{CoG} = ( {X}_{1} \cdot {W}_{1} + {X}_{2} \cdot {W}_{2} + {X}_{3} \cdot {W}_{3} + {X}_{4} \cdot {W}_{4} ) / {W}_{T} \qquad Eq . 2$$

$${Y}_{CoG} = ( {Y}_{1} \cdot {W}_{1} + {Y}_{2} \cdot {W}_{2} + {Y}_{3} \cdot {W}_{3} + {Y}_{4} \cdot {W}_{4} ) / {W}_{T} \qquad Eq . 3$$

It should be noted that the XCoG and YCoG coordinates represent the CoG of the surgical package 300 in a first orientation, such as the bottom portion of the surgical package 300 contacting the load cells 236. The surgical package 300 may be rotated 90° to orient the surgical package 300 in a second orientation, such a a side portion of the surgical package 300 contacting the load cells 236. The XCoG and YCoG coordinates would then represent the CoG of the surgical package 300 in the second orientation. Similarly, the surgical package 300 may be rotated another 90° to orient the surgical package 300 in a third orientation, such as a front portion of the surgical package 300 contacting the load cells 236. The XCoG and YCoG coordinates would then represent the CoG of the surgical package 300 in the third orientation.

Advantageously, the controller 172 may be configured to identify the item within the surgical package 300 based on the load cell data, the total weight (WT) and the CoG coordinates XCoG and YCoG.

In certain embodiments, the controller 172 may be configured to control the gripper 220 to present the surgical package 300 to the load cells 236 of the scale 234 (such as in the first orientation), command the scale 234 to acquire load cell data, and then determine, based on the load cell data, a type of item within the surgical package 300.

More particularly, the controller 172 may be configured to determine, based on the load cell data, the weight (WT) of the surgical package 300 and the center-of-gravity (XcoG and YCoG) of the surgical package 300, and then determine, based on the weight (WT) and the center-of-gravity (XCoG and YCoG), the type of item. For example, the weight (WT) and the center-of-gravity (XCoG and YCoG) for different items may be stored in the instrument library 176, and the controller 172 may compare the weight (WT) and the center-of-gravity (XCoG and YCoG) to the data within the instrument library 176 to determine which item is contained within the surgical package 300.

In certain embodiments, the center-of-gravity (XCoG and YCoG) associated with a single orientation of the surgical package 300 may be stored in the instrument library 176. The controller 172 then compares the weight (WT) and the center-of-gravity (XCoG and YCoG) for the same orientation to the data within the instrument library 176 to determine which item is contained within the surgical package 300.

In other embodiments, the center-of-gravity (XCoG and YCoG) associated with two orientations of the surgical package 300 may be stored in the instrument library 176. The controller 172 then compares the weight (WT) and the center-of-gravity (XCoG and YCoG) for the same orientations to the data within the instrument library 176 to determine which item is contained within the surgical package 300.

In other embodiments, the center-of-gravity (XCoG and YCoG) associated with three orientations of the surgical package 300 may be stored in the instrument library 176. The controller 172 then compares the weight (WT) and the center-of-gravity (XCoG and YCoG) for the same orientations to the data within the instrument library 176 to determine which item is contained within the surgical package 300.

In certain embodiments, the instrument library 176 may store additional information associated with the surgical package 300, such as an opening portion of the surgical package 300, the location of the item within the surgical package 300, and the orientation of the item within the surgical package 300. After identifying the type of item within the surgical package 300, the controller 172 may also be configured to determine (or retrieve) this additional information from the instrument library 176, which may assist in opening the package, as described below.

FIG. 2B is a diagram illustrating an end effector 240 for identifying an item in a surgical package 300, in accordance with embodiments of the present disclosure.

The dual end effector arm 200, the end effector arm 210, the end effector arm 212, and the gripper 220 were discussed above with respect to FIG. 2A.

The end effector 240 is coupled to the end effector arm 212. Generally, the end effector 240 includes a sensor that is configured to transmit sensor data to the controller 172. More particularly, the end effector 240 includes an x-ray source 242, a support art 244, and an x-ray sensor 246. The x-ray source 242 is configured to illuminate the surgical package 300 with an x-ray beam, and the x-ray sensor 246 is configured to transmit x-ray image data to the controller 172.

It should be noted that the x-ray image data represent the surgical package 300 in a first orientation, such as the front side of the surgical package 300 facing the x-ray source 242 and the rear side of the surgical package 300 facing the x-ray sensor. The first orientation may be a plan view of the surgical package 300 rather than a side view.

The surgical package 300 may be rotated 180° to orient the surgical package 300 in a second orientation, such as the rear side of the surgical package 300 facing the x-ray source 242 and the front side of the surgical package 300 facing the x-ray sensor. The x-ray image data would then represent the surgical package 300 in the second orientation. The second orientation may also be a plan view of the surgical package 300 rather than a side view.

Advantageously, the controller 172 may be configured to identify the item within the surgical package 300 based on the x-ray image data.

In certain embodiments, the controller 172 may be configured to control the gripper 220 to present the surgical package 300 to between the x-ray source 242 and the x-ray sensor 246 (such as in the first orientation), command the x-ray source 242 to generate x-rays and the x-ray sensor 246 to acquire x-ray image data, and then determine, based on the x-ray image data, a type of item within the surgical package 300.

In certain embodiments, the x-ray image data associated with a single orientation of the surgical package 300 may be stored in the instrument library 176. The controller 172 then compares the x-ray image data for the same orientation to the data within the instrument library 176 to determine which item is contained within the surgical package 300. This process includes image detection, image comparison, etc.

In certain embodiments, a machine learning (ML) model may be trained to detect a number of different items in surgical packages based on x-ray image training data. The controller 172 executes the ML model to detect the item in the x-ray image data provided by x-ray sensor 246. After the item is detected, the controller 172 then compares (or matches) the detected item to the data stored in the instrument library 176 to determine the type of item within the surgical package 300.

In other embodiments, the x-ray image data associated with two orientations of the surgical package 300 may be stored in the instrument library 176. The controller 172 then compares the x-ray image data for the same orientations to the data within the instrument library 176 to determine which item is contained within the surgical package 300.

As described above, the instrument library 176 may store additional information associated with the surgical package 300, such as an opening portion of the surgical package 300, the location of the item within the surgical package 300, and the orientation of the item within the surgical package 300. After identifying the type of item within the surgical package 300, the controller 172 may also be configured to determine (or retrieve) this additional information from the instrument library 176, which may assist in opening the package, as described below.

FIG. 2C is a diagram illustrating an end effector 250 for identifying an item in a surgical package 300, in accordance with embodiments of the present disclosure.

In certain embodiments, the surgical package 300 includes a tag 301 that has information, such as the type of item contained within the surgical package 300, printed in a fluorescent pigment.

The dual end effector arm 200, the end effector arm 210, the end effector arm 212, and the gripper 220 were discussed above with respect to FIG. 2A.

The end effector 250 is coupled to the end effector arm 212. Generally, the end effector 240 includes a sensor that is configured to transmit sensor data to the controller 172. More particularly, the end effector 250 includes a fluorescent light source (such as an ultra-violet or UV light source) that is configured to illuminate the surgical package 300 with a fluorescent beam, and a camera (the sensor) that is configured to acquire image data in the visible spectrum. The camera is configured to transmit the image data to the controller 172.

The information printed on the tag 301 is not visible when illuminated by light in the visible spectrum. When illuminated by fluorescent light, the information printed on the tag 301 becomes visible, and the camera may acquire an image of the tag 301 and the information printed thereon.

Advantageously, the controller 172 may be configured to identify the item within the surgical package 300 based on the image data.

In certain embodiments, the controller 172 may be configured to control the gripper 220 to present the surgical package 300 to the end effector 250 (such as in the first orientation that presents the tag 301 to the end effector 250), command the fluorescent light source to generate fluorescent light and the camera to acquire image data, and then determine, based on the image data, a type of item within the surgical package 300.

In certain embodiments, an ML model may be trained to detect the information in the tag 301 based on character-based image training data. The controller 172 executes the ML model to detect the information provide in the image data, such as the type of item contained within the surgical package 300. In certain embodiments, the information may also include an opening portion of the surgical package 300, the location of the item within the surgical package 300, and the orientation of the item within the surgical package 300.

In other embodiments, the information only includes the type of item contained within the surgical package 300, and the controller 172 may then compare the type of item to the data stored in the instrument library 176 to determine (or retrieve) the opening portion, the location of the item, and the orientation of the item from the instrument library 176, which may assist in opening the package, as described below.

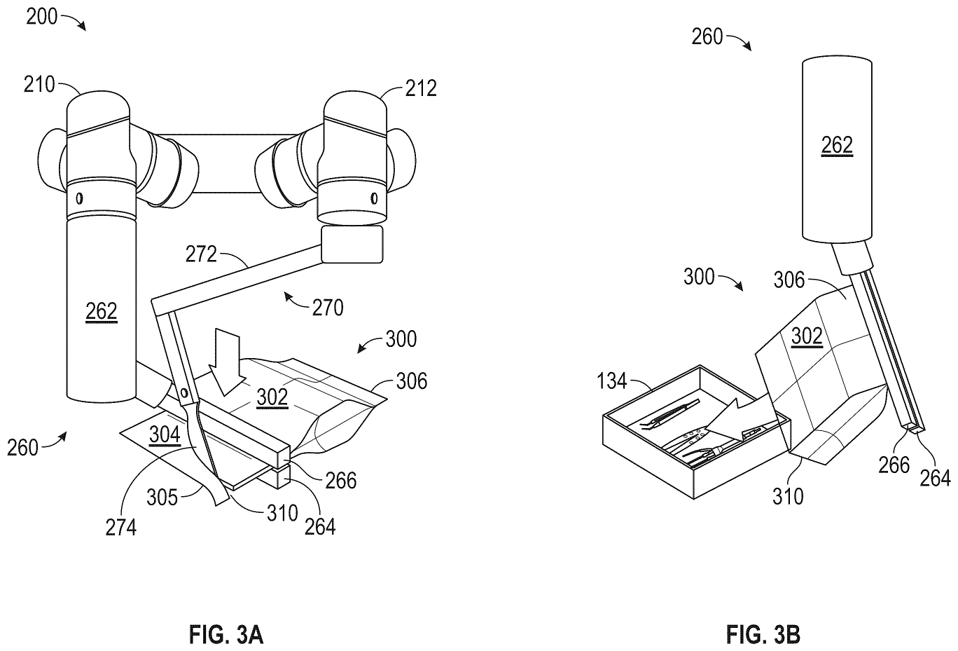

FIG. 3A is a diagram illustrating end effectors 260, 270 for opening a surgical package 300, in accordance with embodiments of the present disclosure.

The surgical package 300 has a body 302, an opening portion 304, and outside edge 305, and an opposite edge 306. The controller 172 may determine the opening portion 304 in several different ways, as discussed above.

In certain embodiments, the dual end effector arm 200 includes an end effector arm 210 and an end effector arm 212. The dual end effector arm 200 is coupled to the end of the robotic arm 118 of setup robot 116, such as the first robotic arm 118 (depicted in FIG. 1B, 1D) or the second robotic arm 118 depicted in FIG. 1D.

The end effector 260 is coupled to the end effector arm 210. The end effector 260 is configured to hold, manipulate, and release the surgical package 300, and is coupled to the controller 172. The end effector 260 includes a body 262, a first clamp arm 264 and a second clamp arm 266. The first clamp arm 264 and the second clamp arm 266 are configured to clamp the surgical package 300 along the entire length of one dimension, such as the width or the length.

The end effector 270 is coupled to the end effector arm 212. Generally, the end effector 270 is configured to open the surgical package 300. More particularly, the end effector 270 includes an articulated arm 272 that is coupled to a cutting tool 274 (such as a knife, a circular saw blade, etc.) that is configured to cut through the material of the surgical package 300 to create the opening 310 in the surgical package 300. The cutting tool 274 may be removable and autoclavable.

In certain embodiments, the controller 172 may be configured to control the end effector 260 to present the surgical package 300 to the end effector 270, control the end effector 270 to open the surgical package 300 based on the type of item, the opening portion 304 of the surgical package 300, and the location of the item, and control the end effector 260 to release the item from the surgical package 300.

For example, the controller 172 may be configured to control the end effector 260 to clamp the surgical package 300 at the opening portion 304, and control the end effector 270 to cut open the surgical package 300 along the outside edge 305 of the opening portion 304. As discussed above, the controller 172 has previously determined the location of the item within the surgical package 300, so the controller 172 may retrieve the dimensions of the item from the instrument library 176 to determine the footprint of the item within the surgical package 300. The controller 172 then compares the footprint of the item to the opening portion 304, and controls the end effector 26 to clamp the opening portion 304 at a location that is not occupied by the item.

In certain other embodiment, the end effector 260 may be replaced by a gripper 220, and a motorized clamp may be mounted to a surface that is proximate to the setup robot 116, such as supply area 132. The motorized clamp is coupled to the controller 172, and performs the same function as the end effector 260. The controller 172 is configured to control the gripper 220 to grasp and present the opening portion 304 of the surgical package 300 to the motorized clamp, control the motorized clamp to clamp the opening portion 304 of the surgical package 300 and control the end effector 270 to open the surgical package 300.

FIG. 3B is a diagram illustrating releasing an item from a surgical package 300, in accordance with embodiments of the present disclosure.

In certain embodiments, the controller 172 is also configured to control the end effector 260 to clamp the surgical package 300 at a different location than the opening portion (such as the opposite edge 306), and rotate the surgical package 300 to release the item through the opening 310 of the surgical package 300 and onto the tray 134.

For those embodiments in which the end effector 260 is replaced by a gripper 220, the controller 172 is configured to control the gripper 220 to grasp the surgical package 300 at a different location than the opening portion (such as the opposite edge 306), and rotate the surgical package 300 to release the item through the opening 310 of the surgical package 300 and onto the tray 134.

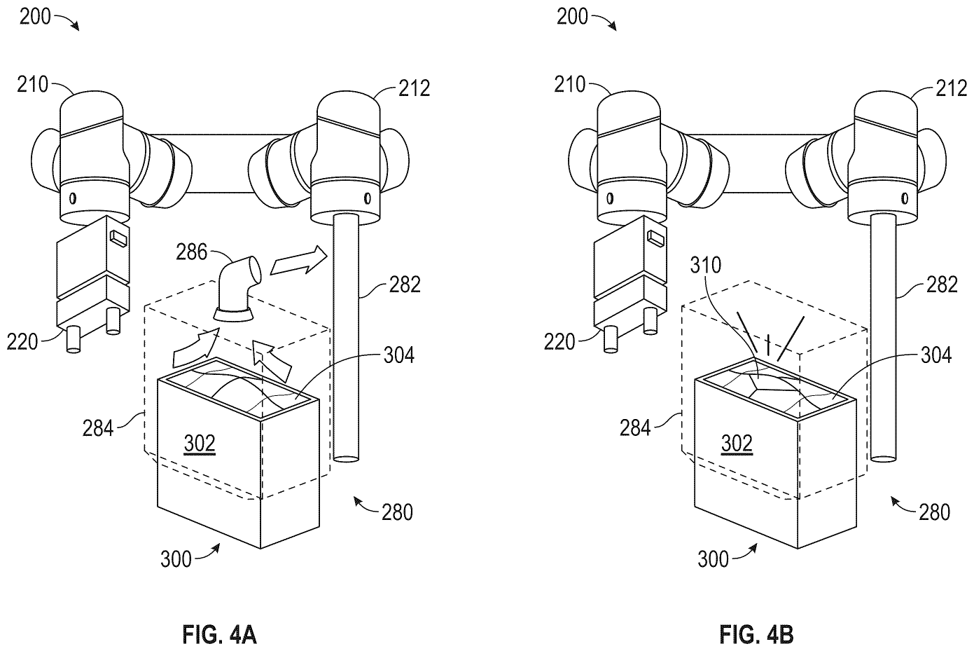

FIG. 4A is a diagram illustrating an end effector 280 for opening a surgical package 300, in accordance with embodiments of the present disclosure.

The surgical package 300 has a body 302 and an opening portion 304. The controller 172 may determine the opening portion 304 in several different ways, as discussed above.

The dual end effector arm 200, the end effector arm 210, and the end effector arm 212 were discussed above with respect to FIG. 3A.

The gripper 220 is an end effector that is coupled to the end effector arm 210. The gripper 220 is configured to hold, manipulate, and release the surgical package 300, and is coupled to the controller 172.

The end effector 280 is coupled to the end effector arm 212. Generally, the end effector 280 is configured to open the surgical package 300. More particularly, the end effector 280 is a vacuum tool that includes a support arm 282 that is coupled to a sterile chamber 284. A vacuum supply port 286 in the sterile chamber 284 is coupled to a vacuum supply line from the console 144.

In certain embodiments, the controller 172 may be configured to control the gripper 220 to present the surgical package 300 to the end effector 280, control the end effector 280 to open the surgical package 300 based on the type of item, the opening portion 304 of the surgical package 300, and the location of the item, and control the gripper 220 to release the item from the surgical package 300 (as depicted in FIG. 3B).

For example, the controller 172 may be configured to control the end effector 280 to seal at least the opening portion 304 within the sterile chamber 284, and control the end effector 280 to open the surgical package 300 using a vacuum generated by the vacuum supply line.

FIG. 4B is a diagram illustrating opening a surgical package 300 using the end effector 280 of FIG. 4A, in accordance with embodiments of the present disclosure.

After the vacuum has been generated for a period of time, the opening 310 forms in the body 302 of the surgical package 300. After the opening 310 is formed, the vacuum may be released, the gripper 220 may remove the surgical package 300 from the sterile chamber 284, and rotate the surgical package 300 to release the item through the opening 310 of the surgical package 300 and onto the tray 134.

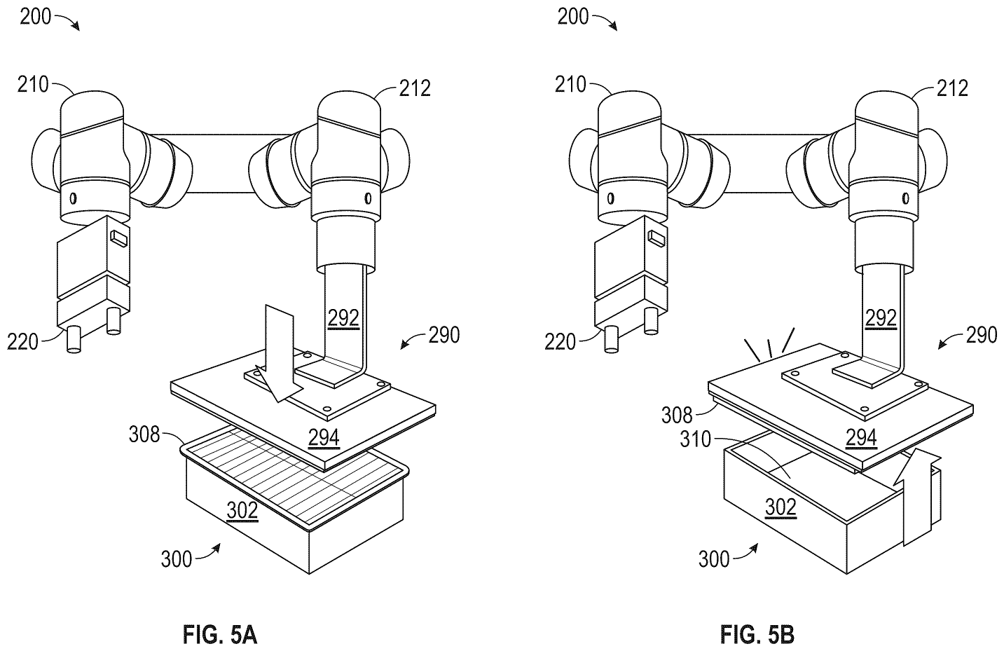

FIG. 5A is a diagram illustrating an end effector 290 for opening a surgical package 300, in accordance with embodiments of the present disclosure.

The surgical package 300 has a body 302 and a cover 308. The opening portion 304 is the cover 308, which is sealed to the body 302 using an adhesive. The controller 172 may determine the opening portion 304 in several different ways, as discussed above.

The dual end effector arm 200, the end effector arm 210, and the end effector arm 212 were discussed above with respect to FIG. 3A.

The gripper 220 is an end effector that is coupled to the end effector arm 210. The gripper 220 is configured to hold, manipulate, and release the surgical package 300, and is coupled to the controller 172.

The end effector 290 is coupled to the end effector arm 212. Generally, the end effector 290 is configured to open the surgical package 300. More particularly, the end effector 290 is a thermal tool that includes a support arm 292 that is coupled to a heat plate 294. An electrical power cable is coupled to the heat plate 294 from the console 144.

In certain embodiments, the controller 172 may be configured to control the gripper 220 to present the surgical package 300 to the end effector 290, control the end effector 290 to open the surgical package 300 based on the type of item, the opening portion 304 of the surgical package 300, and the location of the item, and control the gripper 220 to release the item from the surgical package 300 (as generally depicted in FIG. 3B).

For example, the controller 172 may be configured to control the end effector 290 to couple the heat plate 294 to the cover 308 (using releasable connectors, latches, etc.), control the end effector 290 to increase the temperature of the heat plate 294, and control the end effector 290 to remove the cover 308 from the body of the surgical package 300.

FIG. 5B is a diagram illustrating opening a surgical package 300 using the end effector 290 of FIG. 5A, in accordance with embodiments of the present disclosure.

After the heat has been applied to the cover 308 for a period of time, the adhesive softens and the cover 308 may be removed to form the opening 310 in the body 302 of the surgical package 300. After the opening 310 is formed, the gripper 220 rotate the surgical package 300 to release the item through the opening 310 of the surgical package 300 and onto the tray 134.

In certain embodiments, the controller 172 may be configured to update the inventory 179 stored in memory 173 to reflect not only the number of surgical packages 300 (with type of item) that are opened, but also the items that are actually used during the ophthalmic procedure.

In certain embodiments, a custom surgical pack may comprise a set of surgical packages 300 with various items that is tailored for a specific ophthalmic procedure. While all of the items of the custom surgical pack are removed from the surgical packages 300 and placed on the tray 134 before the ophthalmic procedure, not all of the items may be used during the ophthalmic procedure. Tracking the items from a custom surgical pack that are actually used during the ophthalmic procedure allows the contents of the custom surgical pack to be optimized to reduce waste.

In other words, embodiments of the present disclosure advantageously provide fulfillment and supply chain business intelligence related to custom surgical packs and the ability to realize cost savings by being more precise about the contents of the custom surgical packs.

In certain embodiments, the controller 172 may be configured to weigh certain items before and after the ophthalmic procedure using, for example, end effector 230. A change in the weight or the CoG of an item may indicate that a portion of the item may have been left inside the patient 106.

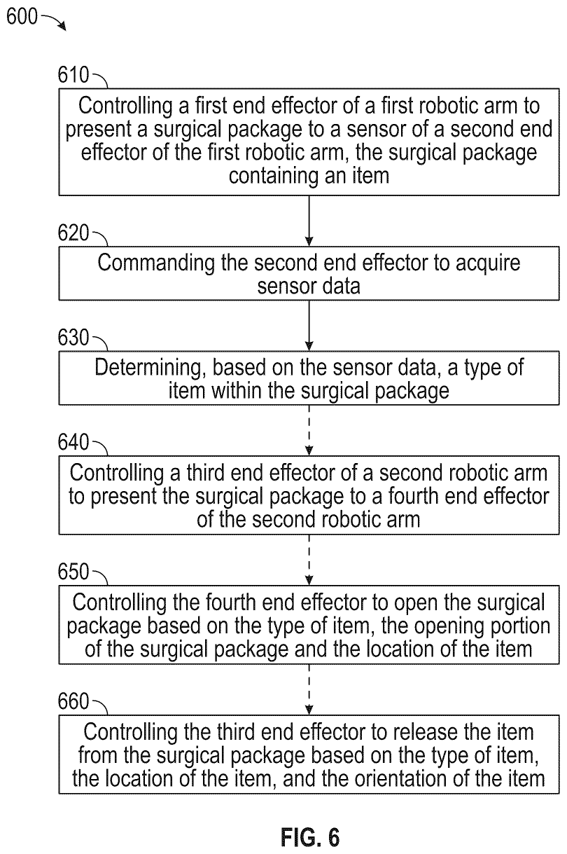

FIG. 6 depicts process an example flow diagram 600 presenting functionality associated with controlling a robot, in accordance with certain embodiments of the present disclosure. In certain embodiments, the blocks 640, 650, 660 may be performed after the blocks 610, 620, 630 are performed, while is certain other embodiments, the blocks 640, 650, 660 may be omitted.

At 610, the controller 172 controls a first end effector (such as the gripper 220) of a first robotic arm 118 to present a surgical package 300 to a sensor (such as the scale 234, the x-ray sensor 246, a camera, etc.) of a second end effector 230, 240, 250 of the first robotic arm 118. The surgical package 300 contains an item.

At 620, the controller 172 commands the second end effector 230, 240, 250 to acquire sensor data (such as load cell data, x-ray image data, image data, etc.).

At 630, the controller 172 determines, based on the sensor data, a type of item within the surgical package 300.

In certain embodiments, the controller 172 also determines, based on the sensor data, an opening portion 304 of the surgical package 300, a location of the item within the surgical package 300, and an orientation of the item within the surgical package 300.

At 640, the controller 172 controls a third end effector (e.g., gripper 220 or end effector 260) of a second robotic arm 118 to present the surgical package 300 to a fourth end effector (e.g., end effectors 270, 280, or 290) of the second robotic arm 118.

At 650, the controller 172 controls the fourth end effector (e.g., end effectors 270, 280, or 290) to open the surgical package 300 based on the type of item, the opening portion of the surgical package and the location of the item.

At 660, the controller 172 controls the third end effector (e.g., gripper 220 or end effector 260) to release the item from the surgical package 300.

### Example Embodiments

In certain embodiments, a system comprises a first robotic arm and a controller. The first robotic arm comprises a first end effector configured to hold and manipulate a surgical package containing an item, and a second end effector comprising a sensor configured to transmit sensor data. The controller is coupled to the first end effector and the second end effector. The controller is configured to control the first end effector to present the surgical package to the sensor of the second end effector, command the second end effector to acquire the sensor data, determine, based on the sensor data, a type of item within the surgical package.

In certain embodiments, a method for controlling a robot comprises controlling a first end effector of a first robotic arm to present a surgical package to a sensor of a second end effector of the first robotic arm, the surgical package containing an item; commanding the second end effector to acquire sensor data; and determining, based on the sensor data, a type of item within the surgical package.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A system, comprising:
a first robotic arm comprising:
a first end effector configured to hold and manipulate a surgical package containing an item, and
a second end effector comprising a sensor configured to transmit sensor data; and
a controller, coupled to the first end effector and the second end effector, the controller configured to:
control the first end effector to present the surgical package to the sensor of the second end effector,
command the second end effector to acquire the sensor data, and
determine, based on the sensor data, a type of item within the surgical package.

2. The system of claim 1, wherein:
the controller is further configured to determine based on the sensor data:
an opening portion of the surgical package;
a location of the item within the surgical package; and
an orientation of the item within the surgical package.

3. The system of claim 2, wherein:
the sensor is a scale comprising a plurality of load cells;
the sensor data is load cell data; and
the controller is further configured to:
determine, based on the load cell data, a weight of the surgical package and a center-of-gravity of the surgical package, and
determine, based on the weight and the center-of-gravity, the type of item, the opening portion of the surgical package, the location of the item, and the orientation of the item.

4. The system of claim 3, wherein the second end effector further comprises a frame to support the surgical package in a center of the plurality of load cells.

5. The system of claim 2, wherein:
the second end effector further comprises an x-ray source configured to illuminate the surgical package with an x-ray beam;
the sensor is an x-ray sensor;
the sensor data is x-ray image data; and
the controller is further configured to:
determine, based on the x-ray image data, the type of item, the opening portion of the surgical package, the location of the item, and the orientation of the item.

6. The system of claim 2, wherein:
the surgical package comprises a tag that includes the type of item printed in a fluorescent pigment;
the second end effector further comprises a fluorescence light source configured to illuminate the tag with non-visible light;
the sensor is a camera;
the sensor data is image data; and
the controller is further configured to:
determine, based on the image data, the type of item, the opening portion of the surgical package, the location of the item, and the orientation of the item.

7. The system of claim 1, wherein:
the first end effector comprises a parallel-jaw gripper, a vacuum gripper, a finger gripper, a pneumatic gripper, an electromagnetic gripper, or a hydraulic gripper; and
the item comprises an instrument, a consumable product, or a structure to be implanted.

8. The system of claim 2, further comprising:
a second robotic arm comprising:
a third end effector configured to hold and manipulate the surgical package, and
a fourth end effector configured to open the surgical package; and
wherein the controller is further configured to:
control the third end effector to present the surgical package to the fourth end effector,
control the fourth end effector to open the surgical package based on the type of item, the opening portion of the surgical package, and the location of the item, and
control the third end effector to release the item from the surgical package.

9. The system of claim 8, wherein:
the third end effector comprises a gripper including opposing clamp arms;
the fourth end effector comprises a cutting tool; and
the controller is further configured to:
control the third end effector to clamp the surgical package at the opening portion,
control the fourth end effector to cut open the surgical package along an outside edge of the opening portion,
control the third end effector to:
clamp the surgical package at a different location than the opening portion, and
rotate the surgical package to release the item from the surgical package onto a tray.

10. The system of claim 8, wherein:
the fourth end effector comprises a vacuum tool including a sterile chamber coupled to a vacuum supply line; and
the controller is further configured to:
control the fourth end effector to seal at least the opening portion within the sterile chamber,
control the fourth end effector to open the surgical package using a vacuum generated by the vacuum supply line, and
control the third end effector to release the item from the surgical package onto a tray.

11. The system of claim 8, wherein:
the opening portion is a cover that is sealed using an adhesive,
the fourth end effector comprises a thermal tool including a heat plate; and
the controller is further configured to:
control the fourth end effector to couple the heat plate to the cover,
control the fourth end effector to increase a temperature of the heat plate,
control the fourth end effector to remove the cover, and
control the third end effector to release the item from the surgical package onto a tray.

12. A method for controlling a robot, comprising:
controlling a first end effector of a first robotic arm to present a surgical package to a sensor of a second end effector of the first robotic arm, the surgical package containing an item;
commanding the second end effector to acquire sensor data; and
determining, based on the sensor data, a type of item within the surgical package.

13. The method of claim 12, further comprising:
determining based on the sensor data:
an opening portion of the surgical package;
a location of the item within the surgical package; and
an orientation of the item within the surgical package.

14. The method of claim 13, wherein:
the sensor is a scale comprising a plurality of load cells;
the sensor data is load cell data; and
the method further comprises:
determining, based on the load cell data, a weight of the surgical package and a center-of-gravity of the surgical package, and
determining, based on the weight and the center-of-gravity, the type of item, the opening portion of the surgical package, the location of the item, and the orientation of the item.

15. The method of claim 13, wherein:
the second end effector further comprises an x-ray source configured to illuminate the surgical package with an x-ray beam;
the sensor is an x-ray sensor;
the sensor data is x-ray image data; and
the method further comprises:
determining, based on the x-ray image data, the type of item, the opening portion of the surgical package, the location of the item, and the orientation of the item.

16. The method of claim 13, wherein:
the surgical package comprises a tag that includes the type of item printed in a fluorescent pigment;
the second end effector further comprises a fluorescence source configured to illuminate the tag with non-visible light;
the sensor is a camera;
the sensor data is image data; and
the method further comprises:
determining, based on the image data, the type of item, the opening portion of the surgical package, the location of the item, and the orientation of the item.

17. The method of claim 13, further comprising:
controlling a third end effector of a second robotic arm to present the surgical package to a fourth end effector of the second robotic arm;
controlling the fourth end effector to open the surgical package based on the type of item, the opening portion of the surgical package and the location of the item; and
controlling the third end effector to release the item from the surgical package.

18. The method of claim 17, wherein:
the third end effector comprises a gripper including opposing clamp arms;
the fourth end effector comprises a cutting tool; and
the method further comprises:
controlling the third end effector to clamp the surgical package at the opening portion,
controlling the fourth end effector to cut open the surgical package along an outside edge of the opening portion,
controlling the third end effector to:
clamp the surgical package at a different location than the opening portion, and
rotate the surgical package to release the item from the surgical package onto a tray.

19. The method of claim 17, wherein:
the fourth end effector comprises a vacuum tool including a sterile chamber coupled to a vacuum supply line; and
the method further comprises:
controlling the fourth end effector to seal at least the opening portion within the sterile chamber,
controlling the fourth end effector to open the surgical package using a vacuum generated by the vacuum supply line, and
controlling the third end effector to release the item from the surgical package onto a tray.

20. The method of claim 17, wherein:
the opening portion is a cover that is sealed using an adhesive,
the fourth end effector comprises a thermal tool including a heat plate; and
the method further comprises:
controlling the fourth end effector to couple the heat plate to the cover,
controlling the fourth end effector to increase a temperature of the heat plate,
controlling the fourth end effector to remove the cover, and
controlling the third end effector to release the item from the surgical package onto a tray.
